(19) United States
(12) Patent Application Publication
(10) Pub. No.: US 2021/0292698 A1
(43) Pub. Date: Sep. 23, 2021

(54) STIRRING DEVICE AND PREPROCESSING DEVICE

(71) Applicants: NATIONAL UNIVERSITY CORPORATION KOBE UNIVERSITY, Kobe-shi, Hyogo (JP); SHIMADZU CORPORATION, Kyoto-shi, Kyoto (JP)

(72) Inventors: Aya TORAI, Kyoto-shi, Kyoto (JP); Yoshihiro MATSUMOTO, Kyoto-shi, Kyoto (JP); Tomohisa HASUNUMA, Kobe-shi, Hyogo (JP)

(73) Assignees: NATIONAL UNIVERSITY CORPORATION KOBE UNIVERSITY, Kobe-shi, Hyogo (JP); SHIMADZU CORPORATION, Kyoto-shi, Kyoto (JP)

(21) Appl. No.: 17/259,612

(22) PCT Filed: Jul. 10, 2019
(86) PCT No.: PCT/JP2019/027325

§ 371 Date: Jan. 12, 2021

(30) Foreign Application Priority Data
Jul. 17, 2018 (JP) 2018-134174

(51) Int. Cl. C12M 1/06

(52) U.S. Cl. CPC C12M 27/02

(57) ABSTRACT

A stirring device 11 includes a stirring mechanism 7 and a control unit 15. The stirring mechanism 7 includes a motor 74 and a pedestal portion 77. In the stirring device 11, the motor 74 repeats forward rotation and reverse rotation alternately under the control of the control unit 15. Then, the pedestal portion 77 repeats turning in one direction and turning in the other direction alternately. As a result, the test tube S repeats the movement in one direction and the movement in the other direction alternately, and the vortex rotating in one direction and the vortex rotating in the other direction are alternately generated in the mixed solution in the test tube S. Then, the mixed solution in the test tube S is stirred to be rotated, and is stirred to be moved in the vertical direction. Therefore, the mixed solution in the test tube S can be efficiently stirred. As a result, the time required for stirring the mixed solution in the test tube S can be shortened.

### TECHNICAL FIELD

The present invention relates to a stirring device for stirring a mixed solution in which a reagent is mixed with cells.

### BACKGROUND ART

A technology has been known in which an analysis such as metabolome analysis is performed by culturing cells of microorganisms and plants in a culture medium in a culture vessel, collecting cells from the culture medium, performing pre-processing, and then supplying the cells to a liquid chromatograph mass spectrometer. In this kind of technology, a sampling apparatus for sampling a culture medium containing cells and a pre-processing apparatus for performing pre-processing on the cells contained in the sampled culture medium are used. The sampling of the culture medium is performed under an aseptic condition (refer to, for example, Patent Document 1 below).

In the pre-processing apparatus, for example, centrifugation of the culture medium, removal of the liquid other than the cells after the centrifugation, supply of a reagent to the cells, stirring of the mixed solution containing the cells and the reagent, and the like are sequentially performed. Among these, the stirring of the mixed solution is performed by, for example, a dedicated stirring device.

As the stirring device, a device for performing the stirring by moving a test tube at a high speed in a state where the test tube in which the mixed solution is housed is held is used. Such a device includes a motor, and a holding mechanism that holds the test tube at a position away from a drive shaft of the motor (position eccentric from the drive shaft). Then, the driving force from the motor is applied to the holding mechanism, so that the test tube is moved at a high speed.

### PRIOR ART DOCUMENT

### Patent Documents

Patent Document 1: Japanese Patent Laid-Open No. 2012-200239

### SUMMARY OF THE INVENTION

### Problems to be Solved by the Invention

In the above-described stirring device, the motor is continuously rotated at a high speed until the stirring of the mixed solution is completed. Therefore, a load is applied to the holding mechanism and a stirring mechanism for a long period of time, which may cause a problem that these mechanisms are damaged. When the time required for the stirring becomes long, there may be a problem that a portion in contact with the test tube is damaged by wear.

The invention has been made in view of the above circumstances, and an object of the invention is to provide a stirring device capable of shortening the time required for the stirring.

### Means for Solving the Problems

(1) A stirring device according to the invention is a stirring device for stirring a mixed solution in which a reagent is mixed with cells. The stirring device includes a pedestal portion, a motor, and a control unit. The pedestal portion receives a bottom portion of a container in which the mixed solution is housed. The motor has a rotation shaft provided at a portion eccentric with respect to the pedestal portion, and turns the pedestal portion by rotating the rotation shaft. The control unit controls rotation of the motor. The control unit performs control such that the motor repeats forward rotation and reverse rotation alternately.

According to such a configuration, in the stirring device, the motor repeats the forward rotation and the reverse rotation alternately under the control of the control unit. Then, the pedestal portion repeats turning in one direction and turning in the other direction alternately. Further, the container received by the pedestal portion repeats the movement in one direction and the movement in the other direction alternately.

As a result, the vortex rotating in one direction and the vortex rotating in the other direction are alternately generated in the mixed solution in the container.

Therefore, the mixed solution in the container can be efficiently stirred.

As a result, the time required for stirring the mixed solution in the container can be shortened.

(2) A pre-processing apparatus according to the invention is a pre-processing apparatus for performing pre-processing on cells. The pre-processing apparatus includes a centrifugation mechanism, a liquid removal mechanism, a reagent supply mechanism, and the stirring device. The centrifugation mechanism centrifuges a container in which a culture medium containing the cells is housed. The liquid removal mechanism removes liquid other than the cells centrifuged in the container by the centrifugation mechanism. The reagent supply mechanism generates a mixed solution by mixing a reagent with the cells in the container after the liquid is removed by the liquid removal mechanism. The stirring device stirs the mixed solution generated by the reagent supply mechanism.

According to such a configuration, in the pre-processing apparatus, the time required for stirring can be shortened. Thus, the whole work time can be shortened.

### Effects of the Invention

According to the invention, in the stirring device, the motor repeats the forward rotation and the reverse rotation alternately under the control of the control unit. Then, the pedestal portion repeats turning in one direction and turning in the other direction alternately. Further, the container received by the pedestal portion repeats the movement in one direction and the movement in the other direction alternately. As a result, the vortex rotating in one direction and the vortex rotating in the other direction are alternately generated in the mixed solution in the container. Therefore, the mixed solution in the container can be efficiently stirred. As a result, the time required for stirring the mixed solution in the container can be shortened.

### BRIEF DESCRIPTION OF THE DRAWINGS

FIG. 1 is a block diagram illustrating a schematic configuration of an automatic pre-processing system including a pre-processing apparatus according to an embodiment of the invention.

FIG. 2 is a perspective view illustrating a configuration of a stirring device according to the embodiment of the invention.

FIG. 3 is a front view of the stirring device of FIG. 2.

FIG. 4 is a sectional view taken along the line A-A of FIG. 3.

### MODE FOR CARRYING OUT THE INVENTION

### 1. Schematic Configuration of Automatic Pre-Processing System

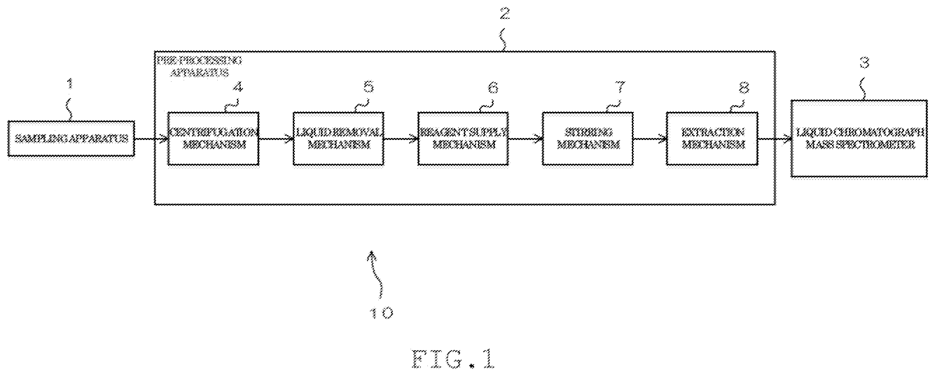

FIG. 1 is a block diagram illustrating a schematic configuration of an automatic pre-processing system 10 including a pre-processing apparatus according to an embodiment of the invention. The automatic pre-processing system 10 is a device for automatically performing pre-processing on an analysis target. In the present embodiment, the analysis target is, for example, a cultured cell, and more specifically, a bacterial cell.

The automatic pre-processing system 10 includes the sampling apparatus 1 and a pre-processing apparatus 2. The metabolites of the cells are extracted from the cells after the pre-processing is performed by the automatic pre-processing system 10, and are supplied to a liquid chromatograph mass spectrometer 3. The liquid chromatograph mass spectrometer 3 is only an example of an analysis device for analyzing an analysis target, and it is also possible to perform an analysis by using another analysis device.

The sampling apparatus 1 is a apparatus for sampling a liquid from a container (culture container). For example, the cells of microorganisms and plants are cultured in a culture medium in a container called a bioreactor, and the culture medium containing the cells in the bioreactor is sampled by the sampling apparatus 1. In the bioreactor, for example, a stirring member that is rotated by using magnetic force, an oxygen concentration sensor for detecting the concentration of dissolved oxygen, and the like are provided, and the cells are cultured in the sampling apparatus 1 by adjusting the dissolved oxygen concentration while stirring the culture medium in the bioreactor.

The pre-processing apparatus 2 performs pre-processing on the cells contained in the culture medium sampled from the bioreactor. In the sampling apparatus 1, the culture medium containing the cells is housed in a test tube as a container (sampling container). The pre-processing apparatus 2 is provided with a centrifugation mechanism 4, a liquid removal mechanism 5, a reagent supply mechanism 6, a stirring mechanism 7, an extraction mechanism 8, and the like, and by each of these mechanisms, the pre-processing is sequentially performed on the cells contained in the culture medium in the test tube.

The centrifugation mechanism 4 centrifuges the test tube in which the culture medium containing the cells is housed. As a result, centrifugal force is applied to the culture medium in the test tube, and the cells (solid) and the liquid other than the cells are separated. Then, the liquid other than the cells centrifuged in the test tube by the centrifugation mechanism 4 is removed by using the liquid removal mechanism 5, and thereby the cells are collected.

The reagent is supplied by the reagent supply mechanism 6, to the test tube after the liquid is removed by the liquid removal mechanism 5. As a result, the reagent is mixed with the cells in the test tube, and a mixed solution is generated. Then, the mixed solution generated by the reagent supply mechanism 6 is stirred by the stirring mechanism 7.

The reagent used in the present embodiment is a reagent for extracting metabolites in the cells, and a suspension in which the metabolites are extracted from the cells is obtained by stirring the mixed solution in which the reagent is mixed with the cells. A part of the suspension obtained in this manner is extracted as an extraction liquid by the extraction mechanism 8, and is supplied to the liquid chromatograph mass spectrometer 3.

### 2. Configuration of Stirring Device

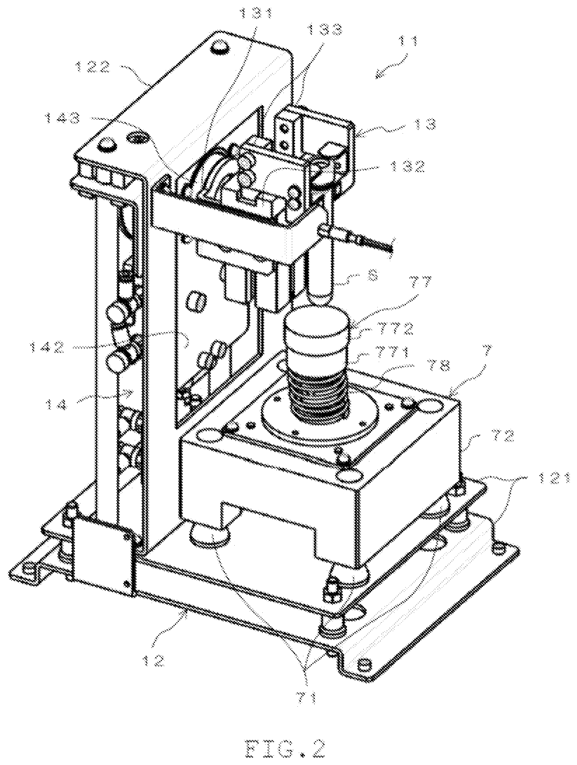

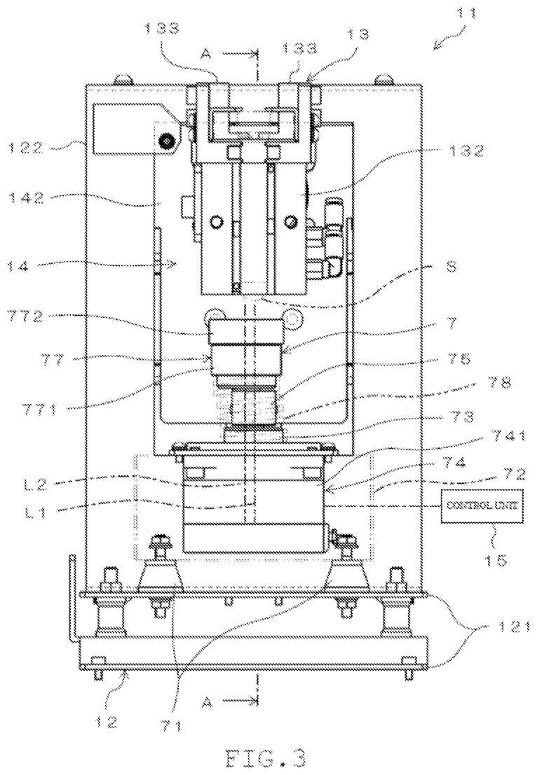

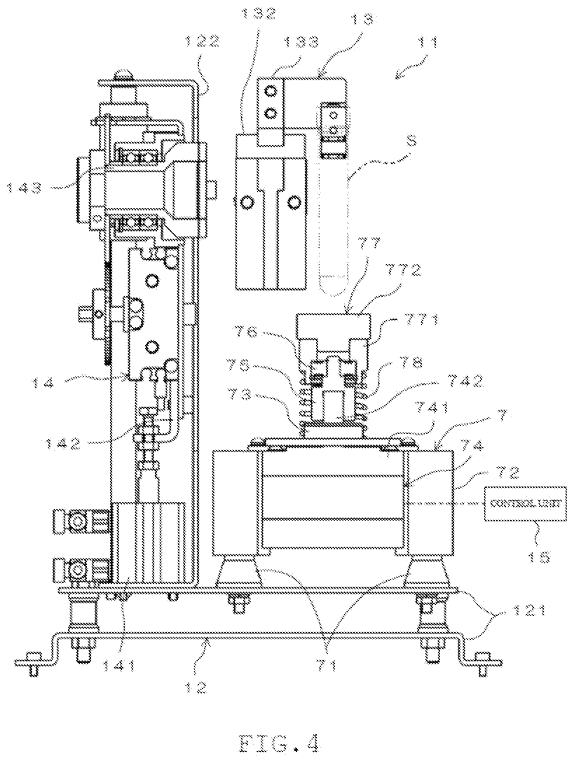

FIG. 2 is a perspective view illustrating a configuration of a stirring device 11 according to the embodiment of the invention. FIG. 3 is a front view of the stirring device 11. FIG. 4 is a sectional view taken along the line A-A of FIG. 3.

The stirring device 11 is provided in the pre-processing apparatus 2 described above. The stirring device 11 includes a frame 12, the above-described stirring mechanism 7, a holding mechanism 13, a movement mechanism 14, and a control unit 15.

The frame 12 is formed in an L shape in a side view, and is made of a metal material. The frame 12 includes a horizontal frame 121 and a vertical frame 122. The horizontal frame 121 is formed in a plate shape, and extends along a horizontal plane. The horizontal frame 121 is fixed to an installation surface (not illustrated). The vertical frame 122 is fixed to the upper surface of the horizontal frame 121. The vertical frame 122 is formed in a plate shape, and extends upward from one end portion of the horizontal frame 121 (left end portion in FIGS. 2 and 4).

The stirring mechanism 7 is fixed on the horizontal frame 121. The stirring mechanism 7 includes a plurality of (four) leg portions 71, a housing 72, a base portion 73, a motor 74, a rotation portion 75, a bearing 76, a pedestal portion 77, and a spring 78.

Each leg portion 71 is fixed to the upper surface of the horizontal frame 121. Each leg portion 71 is formed in a tapered shape that tapers upward. The plurality of (four) leg portions 71 are arranged to be spaced apart from each other.

The housing 72 is fixed on the leg portions 71. The housing 72 is formed in a hollow rectangular parallelepiped shape.

As illustrated in FIGS. 3 and 4, the base portion 73 is fixed to a central portion of the upper surface of the housing 72. The base portion 73 is formed in a cylindrical shape.

The motor 74 is fixed to the housing 72. The motor 74 includes a motor body 741 and a rotation shaft 742. The motor body 741 is fixed in the housing 72. The rotation shaft 742 extends upward from the motor body 741. The rotation shaft 742 is inserted into an opening (not illustrated) formed on the upper surface of the housing 72 and an internal space of the base portion 73. The tip end portion of the rotation shaft 742 is arranged above the housing 72.

The rotation portion 75 is fixed to the rotation shaft 742. The rotation portion 75 is formed in a substantially columnar shape. The rotation portion 75 is formed such that the diameter of the upper portion is small and the diameter of the lower portion is large. A recess that is recessed upward is formed in the lower portion of the rotation portion 75. The rotation shaft 742 of the motor 74 is inserted into the recess of the rotation portion 75.

The bearing 76 is placed on the upper end portion of the rotation portion 75. That is, the upper end portion of the rotation portion 75 is inserted into the internal space of the bearing 76. A part of the inner surface of the bearing 76 is in contact with the peripheral surface of the upper end portion of the rotation portion 75.

The pedestal portion 77 is fixed on the bearing 76. The pedestal portion 77 includes a tubular portion 771, and a plate portion 772.

The tubular portion 771 is formed in a cylindrical shape, and is fixed to the bearing 76. The tubular portion 771 covers the upper surface and the outer peripheral surface of the bearing 76.

The plate portion 772 is fixed to the upper end portion of the tubular portion 771. The plate portion 772 is formed in a disk shape, and is made of a rubber material. The central portion of the lower surface of the plate portion 772 protrudes downward. The lower end portion of the plate portion 772 (protrusion portion at the central portion of the lower surface) is fitted into the inside of the upper end portion of the tubular portion 771.

The spring 78 is a coil spring, and is arranged between the base portion 73 and the pedestal portion 77 (tubular portion 771). Specifically, the lower end portion of the spring 78 is fixed to the outer peripheral surface of the base portion 73, and the upper end portion of the spring 78 is fixed to the outer peripheral surface of the lower end portion of the tubular portion 771. The rotation shaft 742 of the motor 74 and the rotation portion 75 are arranged inside the spring 78.

The holding mechanism 13 is a mechanism for holding a test tube S. The movement mechanism 14 is a mechanism for moving the holding mechanism 13. The movement mechanism 14 is fixed to the vertical frame 122. The holding mechanism 13 is connected to the movement mechanism 14.

As illustrated in FIG. 4, the movement mechanism 14 includes an up-down cylinder 141, a main body portion 142, and a connection portion 143.

The up-down cylinder 141 is fixed to the lower end portion of the vertical frame 122. When air is supplied from an air supply unit (not illustrated), the up-down cylinder 141 moves the cylinder in the vertical direction.

The main body portion 142 is arranged above the up-down cylinder 141. The main body portion 142 constitutes a base part of the movement mechanism 14. The lower end portion of the main body portion 142 is connected to the up-down cylinder 141 (cylinder part in the up-down cylinder 141). The main body portion 142 is moved in the vertical direction together with the cylinder part of the up-down cylinder 141.

The connection portion 143 is attached to the upper end portion of the main body portion 142. The connection portion 143 is formed in a columnar shape extending in the horizontal direction. One end portion of the connection portion 143 (right end portion in FIG. 4) protrudes inward (toward the stirring mechanism 7) from the vertical frame 122.

The holding mechanism 13 includes an arm portion 131, a main body portion 132, and a pair of grip portions 133.

As illustrated in FIG. 2, the arm portion 131 is fixed to one end portion of the connection portion 143 of the movement mechanism 14. The arm portion 131 is formed in a bent plate shape, and extends inward (toward the stirring mechanism 7) from the connection portion 143.

The main body portion 132 is formed in a substantially rectangular parallelepiped shape, and is fixed to the arm portion 131.

Each grip portion 133 is attached to an upper end portion of the main body portion 132. Each grip portion 133 is formed in a plate shape. The one pair of grip portions 133 faces each other in the horizontal direction. The one pair of grip portions 133 can be moved in the opposite direction along the upper surface of the main body portion 132. The grip portion 133 is moved when air is supplied from the air supply unit (not illustrated). Specifically, the test tube S is interposed between the one pair of grip portions 133 by the one pair of grip portions 133 being moved to approach each other in the opposite direction. Further, from this state, the interposed state of the test tube S is released by the one pair of grip portions 133 being moved to be separated from each other in the opposite direction.

The control unit 15 is electrically connected to the motor 74 and the like. The control unit 15 includes, for example, a central processing unit (CPU), and the CPU executes a control program to control the operation of the motor 74 and the like.

In the stirring device 11, the bearing 76 and the pedestal portion 77 are provided at positions eccentric with respect to the rotation shaft 742 of the motor 74. Specifically, as illustrated in FIG. 3, an axis line L2 of the pedestal portion 77 (the bearing 76 and the pedestal portion 77) is deviated from an axis line L1 of the motor 74 (the rotation shaft 742) (is at a position where the axis line L2 does not match the axis line L1).

### 3. Operation of Stirring Device

In the automatic pre-processing system 10, first, the culture medium containing the cells is introduced into the test tube S, and the centrifugation processing is performed on the test tube S. As a result, the culture medium in the test tube S is separated into the cells (solid) and the liquid other than the cells. Further, after the liquid in the test tube S is removed, the reagent is introduced into the test tube S. Then, the test tube S in this state is installed in the stirring device 11.

Specifically, the test tube S is held by the holding mechanism 13 in a state where a cap is put on the upper end portion of the test tube S and the test tube S is along the vertical direction. At this time, the upper end portion of the test tube S is interposed by the grip portions 133 of the holding mechanism 13. When the test tube S is installed in the stirring device 11, as illustrated in FIGS. 3 and 4, the test tube S is located above the pedestal portion 77 of the stirring mechanism 7, and is spaced from the pedestal portion 77.

From this state, the main body portion 142 and the connection portion 143 are moved downward by the operation of the up-down cylinder 141 of the movement mechanism 14. Then, the holding mechanism 13 is moved downward together with the connection portion 143.

Then, the bottom portion of the test tube S comes into contact with the upper surface of the pedestal portion 77 (plate portion 772). When the test tube S comes into contact with the plate portion 772, the operation of the up-down cylinder 141 is stopped, the movement of the main body portion 142 and the connection portion 143 is stopped, and the movement of the holding mechanism 13 is stopped. As a result, a state in which the bottom portion of the test tube S is in contact with the upper surface of the plate portion 772 is maintained.

In this state, the operation of the motor 74 is started under the control of the control unit 15. At this time, the control unit 15 controls the operation of the motor 74 such that the motor 74 repeats forward rotation and reverse rotation alternately.

When the control unit 15 causes the motor 74 (motor body 741) to perform the forward rotation, the rotation shaft 742 of the motor 74 is rotated in one direction about the axis line L1 as illustrated in FIG. 3. Then, the pedestal portion 77 and the bearing 76 are turned in one direction such that the axis line L2 is moved on the circumference of a circle centered on the axis line L1. At this time, the spring 78 is elastically deformed. In this way, the pedestal portion 77 is turned in one direction in accordance with the forward rotation of the motor 74.

Further, when the control unit 15 causes the motor 74 (motor body 741) to perform the reverse rotation, the rotation shaft 742 of the motor 74 is rotated in the other direction about the axis line L1. Then, the pedestal portion 77 and the bearing 76 are turned in the other direction such that the axis line L2 is moved on the circumference of the circle centered on the axis line L1. At this time, the spring 78 is elastically deformed. In this way, the pedestal portion 77 is turned in the other direction in accordance with the forward rotation of the motor 74.

The control unit 15 controls the operation of the motor 74 such that the motor 74 repeats the forward rotation and the reverse rotation alternately. Specifically, the control unit 15 repeats the operation of causing the motor 74 to perform the forward rotation for several seconds and then causing the motor 74 to perform the reverse rotation for several seconds. The time for the motor 74 to perform each of the forward rotation and the reverse rotation is, for example, 1 to 3 seconds.

When the turning of the pedestal portion 77 in one direction and the turning thereof in the other direction are alternately repeated in this way, the test tube S (the bottom portion of the test tube S) repeats the movement in one direction and the movement in the other direction alternately. As a result, the vortex rotating in one direction and the vortex rotating in the other direction are alternately generated in the mixed solution in the test tube S.

When the vortex rotating in one direction and the vortex rotating in the other direction are alternately generated in the mixed solution in the test tube S, the already rotating vortex and the newly generated vortex collide with each other when the rotation of the vortex is switched. As a result, the mixed solution is moved significantly in the vertical direction. In this way, the mixed solution in the test tube S is stirred to be rotated, and is stirred to be moved in the vertical direction.

Therefore, the mixed solution in the test tube S can be efficiently stirred. Then, the time required for stirring the mixed solution in the test tube S can be shortened.

When the stirring processing in the stirring device 11 is completed, the suspension in the test tube S is extracted by the extraction mechanism 8, and is supplied to the liquid chromatograph mass spectrometer 3 (refer to FIG. 1).

### 4. Effects

(1) In the present embodiment, in the stirring device 11, the motor 74 repeats forward rotation and reverse rotation alternately under the control of the control unit 15. Then, the pedestal portion 77 repeats turning in one direction and turning in the other direction alternately.

As a result, the test tube S (the bottom portion of the test tube S) repeats the movement in one direction and the movement in the other direction alternately, and the vortex rotating in one direction and the vortex rotating in the other direction are alternately generated in the mixed solution in the test tube S. Then, the mixed solution in the test tube S is stirred to be rotated, and is stirred to be moved in the vertical direction.

Therefore, the mixed solution in the test tube S can be efficiently stirred.

As a result, the time required for stirring the mixed solution in the test tube S can be shortened.

(2) Further, in the present embodiment, the stirring mechanism 7 (stirring device 11) is provided in the pre-processing apparatus 2.

Therefore, in the pre-processing apparatus 2, the time required for stirring can be shortened. Thus, the whole work time can be shortened.

### 5. Modification Example

In the above embodiment, the case where the mixed solution generated from the culture medium is stirred by the stirring device 11 has been described. However, the stirring device 11 can be used in a case of stirring any other liquid.

### DESCRIPTION OF REFERENCE SIGNS

2 pre-processing apparatus
4 centrifugation mechanism
5 liquid removal mechanism
6 reagent supply mechanism
7 stirring mechanism
11 stirring device
15 control unit
74 motor
77 pedestal portion
741 motor body
742 rotation shaft
771 tubular portion
772 plate portion

1. A stirring device for stirring a mixed solution in which a reagent is mixed with cells, the stirring device comprising:
a pedestal portion that receives a bottom portion of a container in which the mixed solution is housed;
a motor that has a rotation shaft provided at a portion eccentric with respect to the pedestal portion, and turns the pedestal portion by rotating the rotation shaft; and
a control unit that controls rotation of the motor,
wherein the control unit performs control such that the motor repeats forward rotation and reverse rotation alternately.

2. A pre-processing apparatus for performing pre-processing on cells, the pre-processing apparatus comprising:
a centrifugation mechanism that centrifuges a container in which a culture medium containing cells is housed;
a liquid removal mechanism that removes liquid other than the cells centrifuged in the container by the centrifugation mechanism;
a reagent supply mechanism that generates a mixed solution by mixing a reagent with the cells in the container after the liquid is removed by the liquid removal mechanism; and
the stirring device according to claim 1 which stirs the mixed solution generated by the reagent supply mechanism.
